(19) United States
(12) Patent Application Publication
(10) Pub. No.: US 2026/0283537 A1
(43) Pub. Date: Sep. 24, 2026

(54) SYSTEM AND METHOD FOR DIAGNOSING AND ALLEVIATING DEGENERATIVE NEUROLOGICAL DISEASE

(71) Applicant: YOUNGAND INC., Gwangju (KR)

(72) Inventor: Young Uk PARK, Gwangju (KR)

(73) Assignee: YOUNGAND INC., Gwangju (KR)

(21) Appl. No.: 19/370,385

(22) Filed: Oct. 27, 2025

(30) Foreign Application Priority Data
Mar. 18, 2025 (KR) 10-2025-0034930

(51) Int. Cl. A61B 5/00; A61B 5/11; G16H 10/60; G16H 40/67; G16H 50/20

(52) U.S. Cl. CPC A61B 5/4088; A61B 5/0077; A61B 5/1128; A61B 5/4803; A61B 5/4836; A61B 5/7246; A61B 5/7425; G16H 10/60; G16H 40/67; G16H 50/20

(57) ABSTRACT

A system for diagnosing and alleviating degenerative neurological diseases according to an embodiment of the present invention relates to a system for diagnosing and alleviating degenerative neurological diseases which calculates a grade of a user’s degenerative neurological diseases through a provided video and provides a training program corresponding to the calculated grade.

### CROSS-REFERENCE TO RELATED APPLICATION

This application claims priority to and the benefit of Korean Patent Application No. 10-2025-0034930 filed on Mar. 18, 2025, the disclosure of which is incorporated herein by reference in its entirety.

### BACKGROUND

### 1. Field of the Invention

The present invention relates to a system and method for diagnosing and alleviating degenerative neurological diseases, and more particularly, to a system and method for diagnosing and alleviating degenerative neurological diseases which calculates a grade of user’s degenerative neurological diseases through a provided video and provides a training program corresponding to the calculated grade.

### 2. Discussion of Related Art

Degenerative neurological diseases are diseases that cause degenerative changes in neurons of a central nervous system, leading to the loss of functions specific to affected areas and causing various symptoms.

A representative example of the degenerative neurological diseases is dementia syndrome (hereinafter referred to as dementia).

Dementia refers to a syndrome caused by a series of diseases that impair memory and other cognitive functions, leading to difficulties in daily life and social activities.

The most common disease that causes dementia is Alzheimer’s disease, followed by vascular dementia caused by cerebrovascular disease.

Currently, the most effective management of a dementia patient is to make an early diagnosis and begin treatment at an early stage to delay the period in which the dementia patient reaches the severe stages of dementia as much as possible, thereby minimizing the function loss of the dementia patient and alleviating the burden on caregivers of the dementia patient as much as possible.

Even when treatments that may alter the future course of the disease emerge in the future, making the early diagnosis of the dementia and initiating the treatment at an early stage will remain a crucial part of the dementia treatment.

However, dementia is not a disease whose diagnosis may be confirmed solely based on one or two test results and requires careful attention to various clues that indicate memory loss and cognitive decline.

Currently, with the advancement of artificial intelligence technology, studies are also advancing into diagnosing various diseases and conditions based on a user’s behavioral characteristics.

The studies generally employ a method that uses a convolutional neural network (CNN) to extract features and temporal changes from sequential images in a video captured by a user and classify the extracted feature data in order to predict whether the user has a disease or disorder.

In this regard, the applicant disclosed a training device for preventing and diagnosing neurodegenerative diseases, registered as Korean Patent No. 10-2702942 B1 (August 30, 2024).

However, there is also a growing need for technology development that performs a more thorough diagnosis of a user, i.e., a dementia patient, and provides a customized training program accordingly.

### SUMMARY OF THE INVENTION

The present invention is directed to providing a system and method for diagnosing and alleviating degenerative neurological diseases capable of performing a more thorough diagnosis of a user, i.e., a dementia patient, and providing a customized training program accordingly.

Problems to be solved by the present invention are not limited to the above-described objects, and objects that are not mentioned will be clearly understood by those skilled in the art to which the present invention pertains from the present specification and the accompanying drawings.

A system for diagnosing and alleviating degenerative neurological diseases according to an embodiment of the present invention relates to a system for diagnosing and alleviating degenerative neurological diseases which calculates a grade of a user’s degenerative neurological diseases through a provided video and provides a training program corresponding to the calculated grade. The system for diagnosing and alleviating degenerative neurological diseases includes: an input unit that receives a user’s personal information and medical history information, and voice information uttered by the user; a memory unit that stores a first video, which is a video of an expert’s motion and text to be uttered by the user; a camera unit that acquires a second video, which is a video of the user’s motion; a display unit that outputs the first video and the second video; and a control unit that compares accuracy, balance, and reaction speed of the motion output in the second video based on the first video and compares accuracy and pronunciation speed using the voice information to calculate a grade of the user’s degenerative neurological diseases.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic diagram for describing the overall implementation of a system for diagnosing and alleviating degenerative neurological diseases according to an embodiment of the present invention;

FIG. 2 is a block diagram illustrating components of the system for diagnosing and alleviating degenerative neurological diseases according to an embodiment of the present invention;

FIG. 3 is an exemplary diagram illustrating a training program provided by the system for diagnosing and alleviating degenerative neurological diseases according to an embodiment of the present invention;

FIG. 4 is a photograph for describing a first video output on a display unit constituting the system for diagnosing and alleviating degenerative neurological diseases according to an embodiment of the present invention;

FIG. 5 is a photograph for describing the first video and a second video output on the display unit constituting the system for diagnosing and alleviating degenerative neurological diseases according to an embodiment of the present invention;

FIG. 6 is an exemplary diagram for describing that a control unit constituting the system for diagnosing and alleviating degenerative neurological diseases according to an embodiment of the present invention calculates a grade of a user’s degenerative neurological diseases based on the second video;

FIG. 7 is an exemplary photograph illustrating that the login screens are output on the display unit constituting the system for diagnosing and alleviating degenerative neurological diseases according to an embodiment of the present invention;

FIG. 8 is a photograph for exemplarily describing that a guide screen for guiding membership registration is output on the display unit constituting the system for diagnosing and alleviating degenerative neurological diseases according to an embodiment of the present invention;

FIG. 9 is a flowchart illustrating that the login screens are sequentially output on the display unit constituting the system for diagnosing and alleviating degenerative neurological diseases according to an embodiment of the present invention;

FIG. 10 is a flowchart for describing an overall method of diagnosing and alleviating degenerative neurological diseases according to another embodiment of the present invention;

FIG. 11 is a configuration diagram of a training system according to another embodiment of the present invention;

FIG. 12 is a block diagram schematically illustrating a configuration of a training device according to another embodiment of the present invention;

FIG. 13 is a block diagram schematically illustrating a configuration of an external server according to another embodiment of the present invention;

FIGS. 14 and 15 are diagrams illustrating an output screen of a degenerative neurological disease prevention training program according to another embodiment of the present invention; and

FIG. 16 is a flowchart for schematically describing a method of preventing and diagnosing degenerative neurological diseases according to another embodiment of the present invention.

### DETAILED DESCRIPTION OF EXEMPLARY EMBODIMENTS

Hereinafter, detailed embodiments of the present invention will be described in detail with reference to the accompanying drawings. However, it should be noted that the spirit of the present invention is not limited to the embodiments set forth herein and those skilled in the art who understand the present invention can easily accomplish retrogressive inventions or other embodiments included in the spirit of the present invention by the addition, modification, and removal of components within the same spirit, but those are construed as being included in the spirit of the present invention.

A system for diagnosing and alleviating degenerative neurological diseases according to an embodiment of the present invention relates to a system for diagnosing and alleviating degenerative neurological diseases which calculates a grade of a user’s degenerative neurological diseases through a provided video and provides a training program corresponding to the calculated grade. The system for diagnosing and alleviating degenerative neurological diseases includes: an input unit that receives a user’s personal information and medical history information, and the user’s voice information; a memory unit that stores a first video, which is a video of an expert’s motion and voice; a camera unit that acquires a second video, which is a video of the user’s motion; a display unit that outputs the first video and the second video; and a control unit that compares accuracy, balance, and reaction speed of the motion output on the second video based on the first video and compares accuracy and pronunciation speed using the voice information to calculate a grade of the user’s degenerative neurological diseases.

In addition, the control unit may control a login screen to be output on the display unit before the first video is output, the login screen may include an identification screen that identifies a specific body part of the user and an utterance guidance screen that confirms an utterance of the user, and the control unit may acquire login information, which is the user’s image information and the user’s utterance information, through the login screen to calculate the grade of the degenerative neurological diseases.

In addition, the memory unit may store reference information for calculating a numerical value for the login information by comparing the login information, and the control unit may quantify the login information based on the reference information to calculate the grade of the user’s degenerative neurological diseases.

More specifically, the system for diagnosing and alleviating degenerative neurological diseases may further include: a communication unit that transmits the personal information, the medical history information, the second video, the voice information, and the login information to a central server, in which the control unit may control the communication unit to request pre-stored login information from the central server based on the reference information when the numerical value of the login information is greater than or equal to a preset value, and the control unit may calculate the grade of the user’s degenerative neurological diseases based on the pre-stored login information using at least one of a multifactorial variable regression analysis model and an artificial intelligence time series inference model.

Here, the control unit may cause the identification screen to be displayed with priority over the utterance guidance screen when the login screen is output on the display unit, and output the utterance guidance screen on the display unit based on the reference information when a numerical value of the acquired user’s image information is greater than or equal to the preset value.

Furthermore, the memory unit may pre-store a guide screen, which is a user interface (UI) screen for providing guidance on membership registration, and the control unit may control the display unit to output the guide screen when there is no information corresponding to the acquired user’s image information.

A method of diagnosing and alleviating degenerative neurological diseases according to another embodiment of the present invention relates to a method of diagnosing and alleviating degenerative neurological diseases which calculates a grade of a user’s degenerative neurological diseases through a provided video and provides a training program corresponding to the calculated grade. The method includes: receiving, by an input unit, a user’s personal information and medical history information; outputting, by the control unit, a first video, which is a video of an expert’s motion and voice, and a second video, which is a video of the user’s motion acquired from a camera unit, on the display unit; acquiring, by a speaker unit, the user’s voice information based on the first video; calculating, by the control unit a grade of the user’s degenerative neurological diseases based on the second video and the voice information; and outputting, by the control unit, a training video corresponding to the calculated grade on the display unit.

Like reference numerals will be used to designate like components having similar functions throughout the drawings within the scope of the present invention.

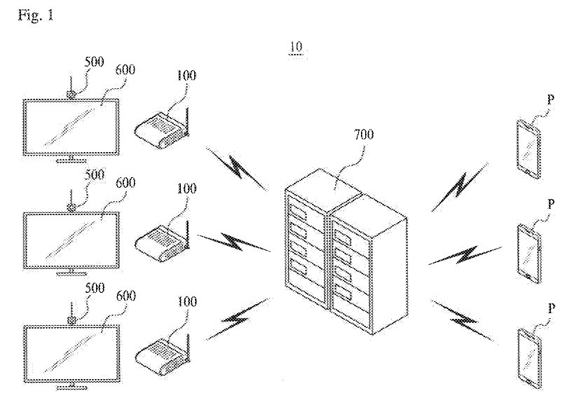

FIG. 1 is a schematic diagram for describing the overall implementation of a system for diagnosing and alleviating degenerative neurological diseases according to an embodiment of the present invention.

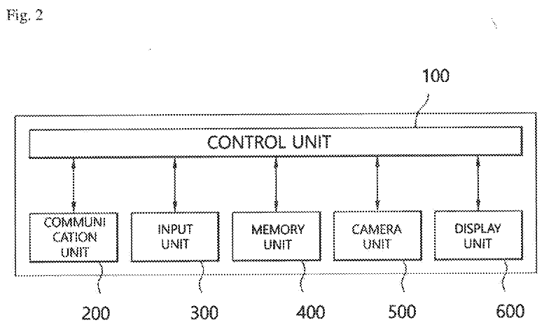

FIG. 2 is a block diagram illustrating components of the system for diagnosing and alleviating degenerative neurological diseases according to an embodiment of the present invention.

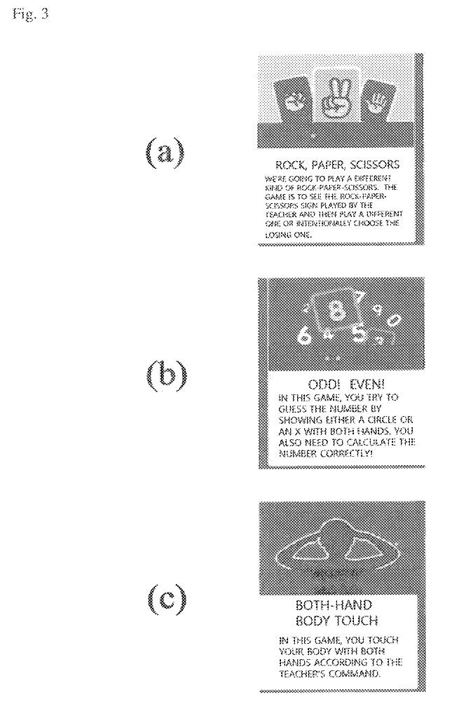

FIGS. 3A to 3C are exemplary diagrams illustrating a training program provided by the system for diagnosing and alleviating degenerative neurological diseases according to an embodiment of the present invention.

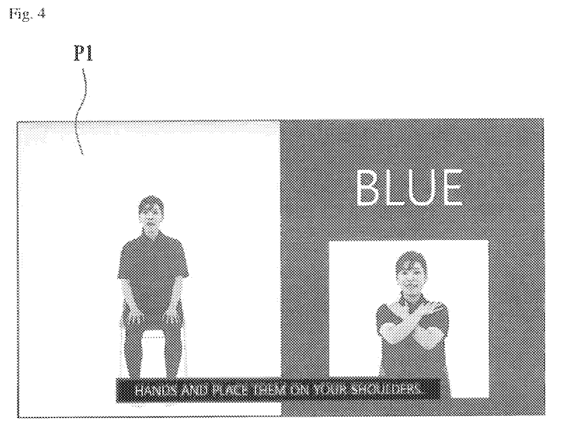

FIG. 4 is a photograph for describing a first video output on a display unit constituting the system for diagnosing and alleviating degenerative neurological diseases according to an embodiment of the present invention.

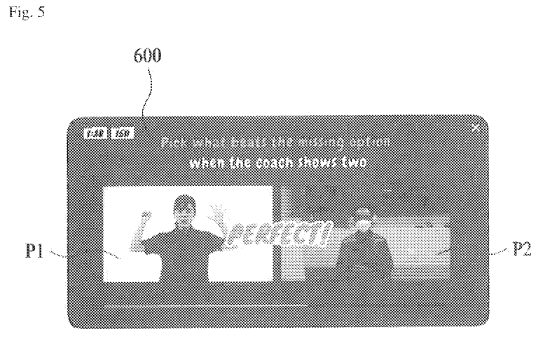

FIG. 5 is a photograph for describing the first video and a second video output on the display unit constituting the system for diagnosing and alleviating degenerative neurological diseases according to an embodiment of the present invention.

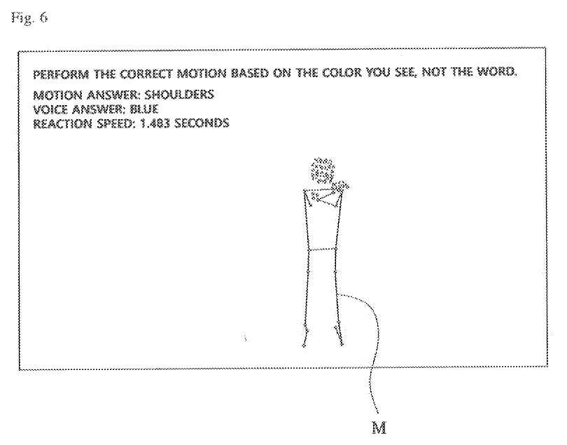

FIG. 6 is an exemplary diagram for describing that a control unit constituting the system for diagnosing and alleviating degenerative neurological diseases according to an embodiment of the present invention calculates a grade of a user’s degenerative neurological diseases based on the second video.

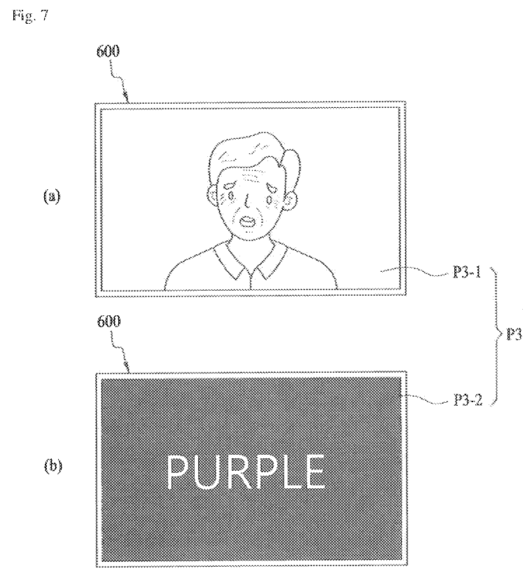

FIGS. 7A and 7B are exemplary photographs illustrating that the login screens are output on the display unit constituting the system for diagnosing and alleviating degenerative neurological diseases according to an embodiment of the present invention.

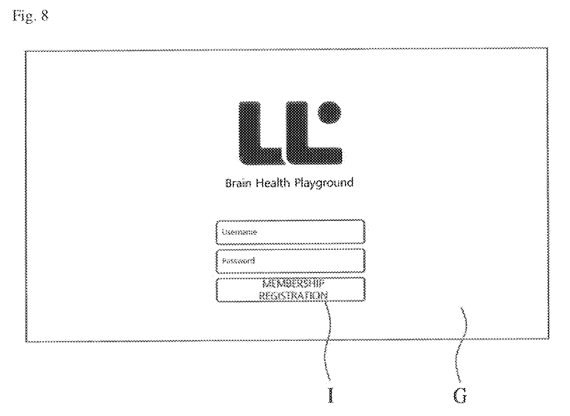

FIG. 8 is a photograph for exemplarily describing that a guide screen for guiding membership registration is output on the display unit constituting the system for diagnosing and alleviating degenerative neurological diseases according to an embodiment of the present invention.

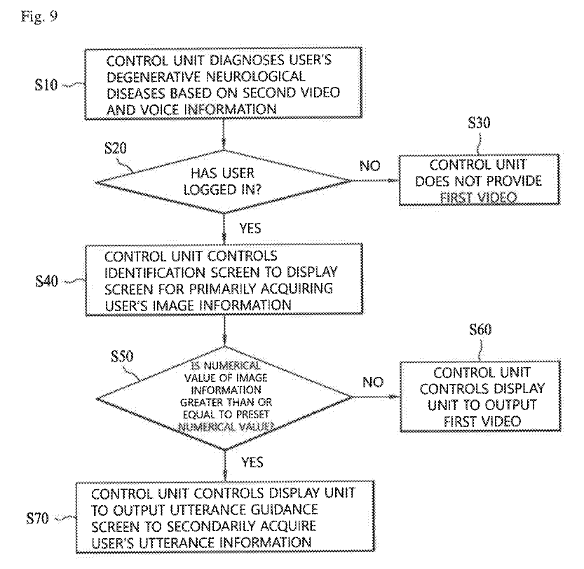

FIG. 9 is a flowchart illustrating that the login screens are sequentially output on the display unit constituting the system for diagnosing and alleviating degenerative neurological diseases according to an embodiment of the present invention.

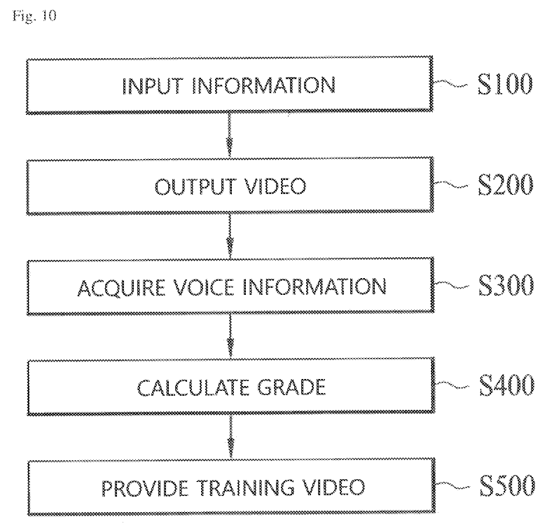

FIG. 10 is a flowchart for describing an overall method of diagnosing and alleviating degenerative neurological diseases according to another embodiment of the present invention.

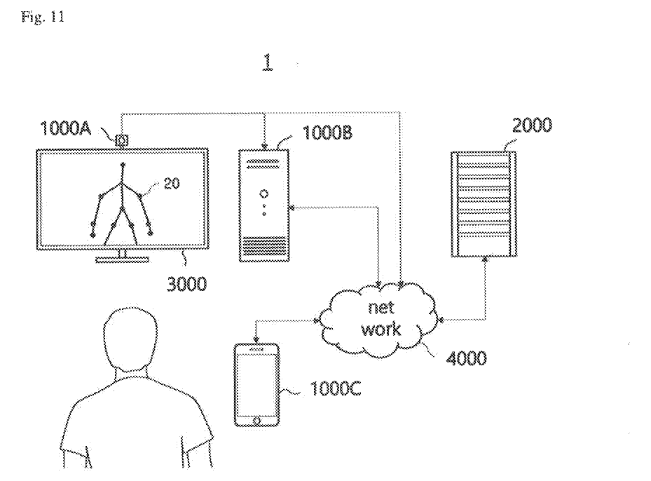

FIG. 11 is a configuration diagram of a training system according to another embodiment of the present invention.

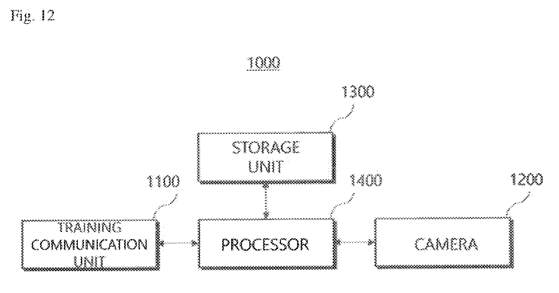

FIG. 12 is a block diagram schematically illustrating a configuration of a training device according to another embodiment of the present invention.

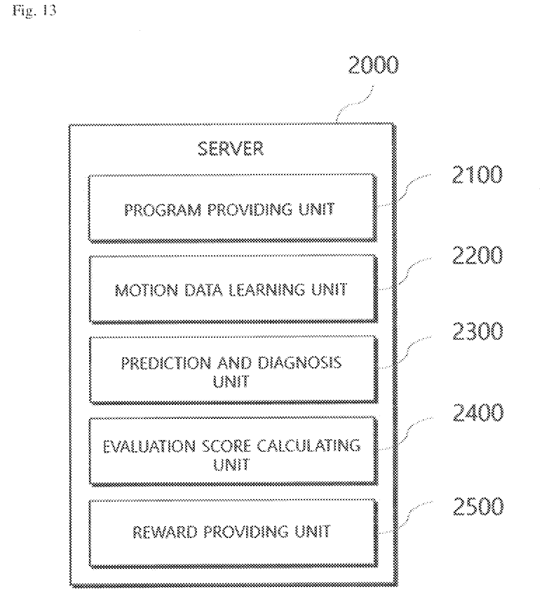

FIG. 13 is a block diagram schematically illustrating a configuration of an external server according to another embodiment of the present invention.

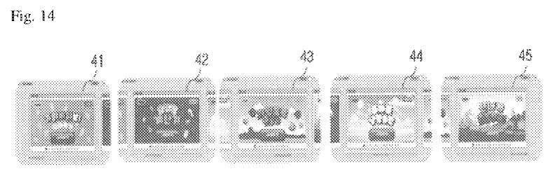

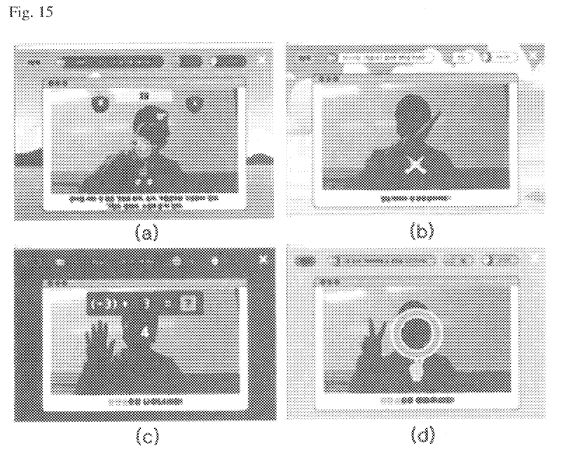

FIGS. 14 and 15 are diagrams illustrating an output screen of a degenerative neurological disease prevention training program according to another embodiment of the present invention.

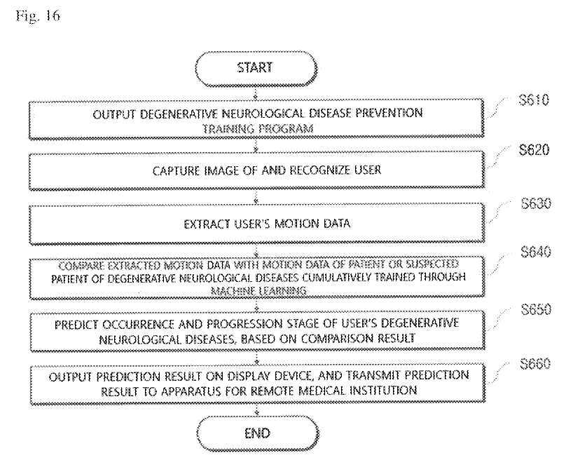

FIG. 16 is a flowchart for schematically describing a method of preventing and diagnosing degenerative neurological diseases according to another embodiment of the present invention.

The attached drawings simplify or omit parts that are less relevant to the technical concept of the present invention or can be easily derived by those skilled in the art, in order to more clearly express the technical concept of the present invention.

Throughout the present specification, when any one part is referred to as being “connected to” another part, it means that the one part and the other part are “directly connected to” each other or are “electrically connected to” each other with still another part interposed therebetween. In addition, when a certain part “includes” a certain component, it means that other components may be further included, rather than excluding other components, unless otherwise stated, and it should be understood that it does not preclude the possibility of addition or presence of one or more other features, numbers, steps, operations, elements, parts, or combinations thereof.

In the present specification, the term “unit” includes a unit implemented by hardware, a unit implemented by software, and a unit implemented by both hardware and software. Further, one unit may be implemented by two or more pieces of hardware, and two or more units may be implemented by one piece of hardware.

In the present specification, some of the operations or functions described as performed by a terminal or a device may be performed instead in a server connected to the corresponding terminal or device. Similarly, some of the operations or functions described as being performed by a server may be performed in a terminal or a device connected to the corresponding server.

Hereinafter, a system and method for diagnosing and alleviating degenerative neurological diseases according to an embodiment of the present invention will be described in detail with reference to FIGS. 1 to 10.

The system 10 for diagnosing and alleviating degenerative neurological diseases (hereinafter referred to as “system”) according to an embodiment of the present invention may refer to a system that diagnoses degenerative neurological diseases (dementia) through a program, such as a video, provided by the system and provides a training program corresponding to the diagnosis result to improve conditions of a user who is a dementia patient.

As illustrated in FIG. 1, the system 10 may be implemented through the user’s terminal P or a device installed in the user’s home and a central server 700.

For example, when the system 10 is installed in the user’s home, the system 10 may include a control unit 100 such as a central processing unit (CPU), a micro processor unit (MPU), or a micro controller unit (MCU), a display unit 600 such as a monitor, and a camera unit 500 that captures the user’s motion and a video of the user’s motion, such as a webcam, etc. When the system 10 is implemented in the form of an application, the system 10 may be implemented through the user’s terminal P, and the control unit 100 may be installed in the home in the form of a set-top box.

However, since the users are generally elderly, the system 10 will be described with the limitation that it is installed in the user’s home.

As illustrated in FIG. 2, the system 10 according to the embodiment of the present invention may include an input unit 300 that receives a user’s personal information and medical history information, and the user’s voice information.

For example, the input unit 300 may be a microphone, and receive the user’s personal information and medical history information, etc., through a guide screen G provided by the system 10 upon membership registration.

As illustrated in FIG. 10, a method (hereinafter referred to as “method”) of diagnosing and alleviating degenerative neurological diseases according to another embodiment of the present invention includes allowing the input unit 300 to receive the user’s personal information and medical history information through an information input operation (operation S100).

The system 10 may provide information necessary for the user’s membership registration through the guide screen G and allow the user to input this information by voice through the input unit 300. Furthermore, by enabling selection of the provided program through a remote device such as a remote control, the system 10 may be more conveniently used by elderly users.

In addition, the system 10 allows the elderly user to use the system 10 more conveniently by making a size of a font output on the guide screen G relatively large so that the font may be recognized more easily by elderly users.

The system 10 may include a memory unit 400 that stores a first video P1, which is a video of a motion and voice of an expert, such as a doctor or a rehabilitation therapist.

For example, as illustrated in FIG. 3, the first video P1 may be a video related to ‘Rock-Paper-Scissors,’ ‘Odd-Even,’ or ‘Both-Hand Body Touch,’ which elderly users may easily follow. When one of the plurality of first videos P1 is selected by the user, the user performs motions following the first video P1 output on the display unit 600 and pronounces text output in the first video P1.

For example, as illustrated in FIG. 4, when the ‘Both-Hand Body Touch’ is selected by the user as the first video P1, the system 10 may output, on the display unit 600, not only the expert’s motion and an explanation for the user to follow the motion, but also the text to be uttered by the user in the video.

In addition, the camera unit 500 may acquire the video of the user’s motion following the first video P1.

Accordingly, the system 10 may simultaneously output not only the first video P1 but also the second video P2, which is the video of the user’s motion, on the display unit 600, as illustrated in FIG. 5.

According to another embodiment of the present invention, as illustrated in FIG. 10, after the information input operation (operation S100), the control unit 100 performs a video output operation (operation S200) to enable the first video P1, which is the video of the expert’s motion and voice, and the second video P2, which is the video of the user’s motion acquired from the camera unit, to be output on the display unit 600.

In addition, the system 10 may receive, from the user through the input unit 300, voice information for the text output from the first video P1.

As illustrated in FIG. 10, the method of another embodiment of the present invention may include, after the video output operation (operation S200), allowing the input unit 300 to acquire the user’s voice information based on the first video P1 through a voice information acquisition operation (operation S300).

Accordingly, the user may repeat a process of following a motion through the training video which is the first video P1 and uttering text output in the first video P1 to alleviate dementia symptoms, and the system 10 may perform a more thorough diagnosis of the user’s degenerative neurological diseases through the second video P2 and the voice information.

More specifically, as illustrated in FIG. 6, the control unit 100 constituting the system 10 compares the accuracy, balance, and reaction speed of the motion output in the second video P2 according to the preset value based on the first video, and compares the accuracy and pronunciation speed using the voice information to calculate a grade for the user’s degenerative neurological diseases, thereby more thoroughly diagnosing the user’s degenerative neurological diseases (operation S10).

The control unit 100 may, for example, use a MobileNet model to detect a preset landmark M of the user’s body and extract motion data by extracting body coordinates, including the user’s face and palms, based on the detected landmark M.

In addition, the control unit 100 may analyze the user’s motion data extracted from the second video P2 based on machine learning to predict the occurrence and progression stages of the user’s degenerative neurological diseases.

Specifically, the central server 700 may store motion data of various users according to a degenerative neurological disease prevention training program that has been cumulatively trained through machine learning, and matching data on the actual occurrence and progression stages of the user’s degenerative neurological diseases.

Accordingly, the control unit 100 may compare the motion data of the user extracted from the second video P2 with motion data of patients with degenerative neurological diseases or patients suspected of having degenerative neurological diseases that has been pre-trained and stored in the central server 700, and diagnose, based on the comparison results, the occurrence of the user’s degenerative neurological diseases, their types (Alzheimer’s disease, vascular dementia, Parkinson’s disease, tremors, chorea, cerebellar degenerative disease, amyotrophic lateral sclerosis, multiple sclerosis, etc.), and their progression stages.

In addition, the control unit 100 may compare the accuracy and pronunciation speed with the pre-stored reference data based on the received voice information and calculate the grade of the user’s degenerative neurological diseases according to the preset value.

As a result, the control unit 100 may calculate the grade of the user’s degenerative neurological diseases according to the preset value, based on the second video P2 and the voice information, and output the first video P1 with the difficulty corresponding to the user’s grade as the training video, thereby alleviating the user’s degenerative neurological diseases.

As illustrated in FIG. 10, the method according to another embodiment of the present invention includes: after the voice information operation (operation S30), a grade calculation operation (operation S400) in which the control unit 100 calculates the grade of the user’s degenerative neurological diseases based on the second video and the voice information; and after the grade calculation operation (operation S400), a training video providing operation (operation S500) in which the control unit 100 provides the first video P1 with the difficulty corresponding to the calculated grade of the user as the training video, thereby alleviating the user’s degenerative neurological diseases.

Meanwhile, the control unit 100 may control a login screen P3 to be output on the display unit 600 before the first video P1 is output.

More specifically, the control unit 100 may identify users who have previously registered for membership through the login screen P3 so that it may provide the first video P1 with the difficulty corresponding to the calculated user level as the training video (operation S20).

For example, the control unit 100 may identify the user through the identification screen P3-1, which is a screen identifying a specific part of a user’s face, as illustrated in FIG. 7A (operation S30)

In addition, after the identification screen P3-1 is displayed, the system 10 may output an utterance guidance screen P3-2 that confirms the user’s utterance, thereby recognizing the voice uttered by the user.

In addition, the memory unit 400 may pre-store reference information with which the login information, i.e., the image information, and the utterance guidance screen with the utterance information uttered by the user, may be compared.

For example, the control unit 100 may generate data on a movement of pre-designated facial landmark, a degree of facial asymmetry, gaze movement, etc., through the facial recognition information, and convert the utterance information using Mel-frequency Cepstral coefficients (MFCCs) to determine pronunciation accuracy, pronunciation speed, whether the utterance matches the contextual flow, etc.

Accordingly, the control unit 100 not only identifies the user through the login screen P3, but also applies at least one of a multifactorial regression analysis model and an artificial intelligence time-series inference model, which are the known models, to the image information and utterance information, thereby accumulating the login information generated upon the user login and uses the accumulated login information as the training data.

The reference information may be image information from when the user was normal in the past, or image information and utterance information of a normal person.

In addition, as may be seen in FIG. 2, the system 10 may further include a communication unit 200 that transmits the personal information, the medical history information, the second video, the voice information, and the login information to the central server 700.

Here, the control unit 100 may control the communication unit 200 to request login information, i.e., past login information, pre-stored in the central server 700, based on the reference information, when the numerical value of the login information is less than a preset value (e.g., the dementia symptoms are lessening), and generate data on the user’s dementia progression over time through an artificial intelligence time series inference model and provide the generated data to the user.

On the other hand, the control unit 100 may control the communication unit 200 to request login information, i.e., past login information, pre-stored in the central server 700, even when the value of the login information is greater than or equal to the preset value (e.g., the dementia symptoms are worsening), and generate data on the user’s dementia progression over time through an artificial intelligence time series inference model and provide the generated data to the user.

That is, the system 10 may utilize the second video, the voice information, and the login information to more thoroughly diagnose the user’s degenerative neurological diseases and further alleviate the user’s dementia symptoms by providing the training video corresponding to the user’s grade.

Furthermore, the system 10 may receive information, such as user’s sex, education level, residence, and medical history, from the user in advance, and allow the diagnosis of the user’s degenerative neurological diseases to be performed more accurately by including the corresponding factor in the multivariate regression analysis performed by the control unit 100.

Meanwhile, the control unit may cause the identification screen P3-1 to be output with priority over the utterance guidance screen P3-2 when the login screen P3 is output on the display unit (operation S40), and when the numerical value of the user’s acquired image information is greater than or equal to the preset value (indicating that the dementia symptoms are not alleviated) (operation S60), based on the reference information for the image information, cause the utterance guidance screen P3-2 to be output on the display unit 600 (operation S70), thereby more thoroughly diagnosing the degenerative neurological diseases through the image information and the utterance information.

In addition, based on the reference information for the image information, the system 10 prevents the utterance guidance screen P3-2 from being output on the display unit 600 when the numerical value of the user’s acquired image information is below the preset value (indicating alleviation of dementia symptoms), thereby reducing the overload of the system 10.

In addition, as illustrated in FIG. 8, the memory unit 400 constituting the system 10 may pre-store the guide screen G, which is the UI screen for guidance on the membership registration.

The guide screen G is manufactured to enable the membership registration using only voice, for example, through the input unit 300 such as a microphone, thereby enabling elderly users to more conveniently perform the membership registration.

Here, when there is no information corresponding to the user’s image information acquired through the login screen P3, i.e., when the image acquired from the user may not be identified, the control unit 100 determines that the user is not registered with the system 10, and controls the display unit 600 to output the guide screen G, thereby enabling elderly users to use the system 10 more easily.

That is, the system 10 diagnoses the user’s degenerative neurological diseases more thoroughly through the second video P2, the voice information, and the login information, and provides the training video based on the diagnosis result, thereby more effectively alleviating the user’s degenerative neurological diseases.

In addition, the system 10 causes text output in the first video P1 to be output in a relatively larger size and allows users to select the membership registration and the first video P1 using a microphone via the guide screen G, thereby reducing the discomfort that may occur when elderly users use the system 10.

Hereinafter, a system 10 for diagnosing and alleviating degenerative neurological diseases according to another embodiment of the present invention will be described in detail with reference to FIGS. 11 to 16.

Hereinafter, the system 10 for diagnosing and alleviating degenerative neurological diseases may be referred to as a training system 1.

FIG. 11 is a configuration diagram of the training system 1 according to another embodiment of the present invention.

As illustrated in FIG. 11, the training system 1 of the present invention includes a training device 1000, an external server 2000, and a display device 3000. According to the embodiment, the training device 1000 may be implemented as a webcam 1000A attached to an upper end of a display device 3000, a desktop main body 1000B connected to the display device 3000, or a user portable terminal 1000C such as a smartphone.

The training device 1000 and the external server 2000 may be connected to each other via a network 4000, and the training device 1000 may be implemented in plurality for each user.

The network 4000 may operate in a wired or wireless manner. The communication method of the network 4000 is not limited, and any communication method utilizing a communication network (e.g., a mobile communication network, a wireless online network, a broadcasting network) that the network 4000 may include may be utilized.

For example, the network 4000 may be implemented with one or more of a wide area network (WAN), a metropolitan area network (MAN), a local area network (LAN), or a combination thereof. In addition, the network 4000 may be implemented with a wireless Internet technology such as a wireless LAN (WLAN) (WiFi), Wireless Broadband (WiBro), and/or World Interoperability for Microwave Access (WiMAX), a mobile communication technology such as code division multiple access (CDMA), Global System for Mobile Communication (GSM), Long Term Evolution (LTE), and/or LTE-Advanced, and/or a vehicle to everything (V2X) communication technology such as a vehicle to infra (V2I).

Meanwhile, the training device 1000 according to another embodiment of the present invention may be implemented as the webcam 1000A. The webcam 1000A is a device that generates image information obtained by capturing an image of a user by connecting to the display device 3000 or a desktop computer 1000B connected to the display device 3000 and is installed and operated at each user’s location and captures each user image to generate user image information. Such a webcam 1000A may be separately connected to a USB device of the display device 3000 or the desktop computer main body 1000B or may be provided in a form in which the webcam 1000A is embedded and operated in a device such as a laptop.

However, according to the embodiment, the training device 1000 may be implemented as the desktop computer 1000B connected to the display device 3000, or as the portable terminal 1000C such as a smartphone, and may be implemented in plurality for each user.

In addition, the training device 1000 according to another embodiment of the present invention may be implemented in various forms such as a tablet, an e-book terminal, a digital broadcasting terminal, a personal digital assistant (PDA), a portable multimedia player (PMP), an MP3 player, a digital camera, and a navigation device, in addition to the above-described forms.

Meanwhile, the external server 2000 according to another embodiment of the present invention has the same configuration as a typical web server in terms of hardware and may include program modules that are implemented in various languages, such as C, C++, Java, Visual Basic, and Visual C in terms of software, to perform various functions. In addition, the external server 2000 may be implemented using various web server programs variously provided according to operating systems such as DoS, Windows, Linux, Unix, Macintosh, Android, and iOS on general server hardware.

The external server 2000 may be implemented to include a database or may be implemented independently of the database. When implemented independently of the database, the external server 2000 may be connected to the database in a wired or wireless manner to exchange files.

The display device 3000 is an electronic device that outputs and displays various pieces of information, including content. In particular, the display device 3000 may output and display the degenerative neurological disease prevention training program. The display device 3000 may be implemented with a liquid crystal display (LCD) panel, organic light emitting diodes (OLEDs), etc., and may also include a driving circuit, a backlight unit, etc., and may be implemented in forms such as an a-Si TFT, a low temperature poly silicon (LTPS) TFT, and an organic TFT (OTFT).

The specific operation of the training system 1 will be described below with reference to FIGS. 12 and 13.

FIG. 12 is a block diagram schematically illustrating a configuration of a training device according to another embodiment of the present invention.

As illustrated in FIG. 12, the training device 1000 includes a training communication unit 1100, a camera 1200, a storage unit 1300, and a processor 1400.

The training communication unit 1100 is configured to communicate with the external server 2000 and a medical institution device (not illustrated). The training communication unit 1100 is designed to include a communication module capable of communicating via the network 4000 described in FIG. 11. The communication method has already been described in FIG. 11, so a detailed description thereof will be omitted.

Meanwhile, the medical institution device is a remote electronic device that receives motion data transmitted from a user or prediction results regarding the occurrence and progression stages of the degenerative neurological diseases, classifies and stores the motion data or prediction results for each patient, and automatically analyzes the motion data or prediction results to generate treatment results or summon medical staff.

The camera 1200 is configured to obtain video data on a user in real time by capturing an image of a predetermined area in front of the user.

The storage unit 1300 may store various pieces of information and programs necessary for the operation of the training device 1000 and temporarily store various input/output data.

In particular, the storage unit 1300 stores video data acquired from the camera 1200 and may record the degenerative neurological disease prevention training program and data related thereto.

The storage unit 1300 may be implemented as one of a hard disk drive (HDD), a solid state drive (SSD), a dynamic random access memory (DRAM), a static random access memory (SRAM), a ferroelectric random access memory (FRAM), and a flash memory, and may also be implemented as a storage device capable of recording various types of data.

The processor 1400 controls the overall operation of the training device 1000.

The processor 1400 may execute various programs, including the degenerative neurological disease prevention training program. The processor 1400 may include a single core or multiple cores (dual core, quad core, octa core, etc.) and may also be referred to as a controller, a microcontroller, a microprocessor, a CPU, an MPU, an MCU, etc.

The training device 1000 according to another embodiment of the present invention may further include a training input unit 1500.

The training input unit 1500 is configured to detect user interaction for controlling the overall operation of the training device 1000 and may receive various pieces of user information.

Here, the user information may include the user’s sex, age, family history, pre-existing diseases, medical history, and drinking history.

Meanwhile, the processor 1400 may control the display device 3000 connected to the training device 1000 to output the degenerative neurological disease prevention training program stored in the storage unit 1300.

In this case, the processor 1400 may capture an image of a user located in front of the display device 3000 and recognize the user via the camera 1200. In addition, according to an embodiment, the processor 1400 may automatically recognize the sex and age of the user whose image is captured by the camera 1200 through the image analysis. Since the technology for recognizing or identifying the user whose image is captured is well known in the relevant technical field, detailed descriptions thereof will be omitted.

While the degenerative neurological disease prevention training program is output through the display device 3000, the processor 1400 may extract motion data corresponding to the user’s commands in the corresponding program or a game being played.

Here, the motion data may include at least one of the motion accuracy, body balance, reaction time, duration, rhythm, and voice accuracy according to the motion commands of the degenerative neurological disease prevention training program.

Here, the degenerative neurological disease prevention training program may be based on a synapsology brain activation program.

The synapsology is a brain activation training method developed by Renaissance, a Japanese fitness center chain. The synapsology is a training approach that activates the brain by continuously changing (spicing up) stimuli or cognitive function stimuli input through a sensory organ by basic motions such as Rock-Paper-Scissors or ball rotation and responding to those stimuli.

For example, the basic motion of the “Fingertip Touch” game is performed as follows: For the left hand, the thumb touches the fingertips in the order of the index finger, the middle finger, the ring finger, and the little finger, and for the right hand, the thumb touches the fingertips in the order of the little finger, the ring finger, the middle finger, and the index finger. This is performed while simultaneously counting from 1 to 10.

The first spice up motion is performed by switching starting fingers and starts with the little finger for the right hand and starts with the index finger for the left hand.

The second spice up motion is performed without looking at the fingertips and starts with the little finger for the right hand and starts with the index finger for the left hand.

As another example, the basic movement of the “Muk-Jji-Ppa & Rock-Paper-Scissors” game is that the left hand moves in the order of “Muk-Jji-Ppa.” The right hand moves its hand in the order of “Ppa-Muk-Jji,” which beats the left hand. In this case, the motion is performed while saying “Ppa-Muk-Jji,” which beats the left hand.

For the first spice up motion, the left hand moves its hand in the order “Ppa-Muk-Jji,” and the right hand moves its hand in the order “Muk-Jji-Ppa.” Both motions are performed while simultaneously saying “Muk-Jji-Ppa,” which beats the right hand.

For the second spice up motion, “Muk-Jji-Ppa” is replaced with “Rock-Scissors-Paper” while the first spice up motion is performed.

Meanwhile, for the “Alternative Rock-Paper-Scissors” game which is the synapsology program played with multiple participants including the instructor, when the instructor says “Rock-Paper-Scissors” and presents one of Muk-Jji-Ppa, the participants look at what the instructor has played and then play a different Rock-Paper-Scissors move than the instructor while saying it.

For the first spice up motion, the instructor plays one of Scissors-Rock-Paper while saying “Scissors-Rock-Paper.” The participant plays Rock-Paper-Scissors later while saying the Scissors-Rock-Paper which beats the instructor.

For the second spice up motion, the instructor plays one of Scissors-Rock-Paper while saying “Scissors-Rock-Paper.” The participant plays Scissors-Rock-Paper later while saying the Scissors-Rock-Paper which loses to the instructor.

For the “Both-Hand Body Touch” game which is another Synapsology program played by multiple participants including the instructor, the instructor gives instructions by saying one of numbers 1 to 4. The participant performs a motion corresponding to that number (1: head touch, 2: shoulder touch, 3: back touch, 4: thigh touch) while saying the given number.

The first spice up motion changes the instructed number into a color (1: red, 2: blue, 3: yellow, 4: green). The instructor gives the instruction while saying a color. The participant performs the motion corresponding to that color while saying the instructed color.

The second spice up motion changes the instructed number into a name of an object, etc.

Synapsology brain activation program screens according to another embodiment of the present invention are as illustrated in FIGS. 14 and 15.

Referring to FIG. 14, a user may select one of multiple synapsology brain activation programs 41 to 45 output through the display device 3000 and perform training.

When any one of the programs is selected, as illustrated in FIGS. 15A to 15D, a captured video of the user is displayed on the display device 3000, and the motion commands are given or the game progresses by displaying content on the screen or outputting voice commands. The user’s body and motion are recognized to extract the motion data.

In another embodiment of the present invention, the processor 1400 may use a MobileNet model to detect the preset landmark on the user’s body and extract body coordinates, including the user’s face and palms, based on the detected landmark to extract the motion data. Here, the MobileNet model may be a convolution neural network (CNN) structure designed for use in the locations where computer performance is limited or battery performance is critical.

Here, as illustrated in FIG. 11, the landmark may include each joint part 20 of the user.

The processor 1400 may analyze the user’s extracted motion data through the machine learning to predict the occurrence and progression stages of the user’s degenerative neurological diseases.

Specifically, the external server 2000 may store motion data of various users according to a degenerative neurological disease prevention training program that has been cumulatively trained through the machine learning, and matching data on the actual occurrence and progression stages of the user’s degenerative neurological diseases.

The actual occurrence and progression stages of the user’s degenerative neurological diseases may be obtained by receiving such diagnostic information from the medical institution device of the medical institution when a user suspected of having degenerative neurological diseases later visits the medical institution and is diagnosed with degenerative neurological diseases.

The processor 1400 may compare the extracted motion data with the motion data of the patients with degenerative neurological diseases or the patients suspected of having the degenerative neurological diseases that has been pre-trained and stored in the external server 2000, and predict, based on the comparison results, the occurrence of the user’s degenerative neurological diseases, their types (Alzheimer’s disease, vascular dementia, Parkinson’s disease, tremors, chorea, cerebellar degenerative disease, amyotrophic lateral sclerosis, multiple sclerosis, etc.), and their progression stages.

According to various embodiments of the present invention, a machine learning-based motion data learning and classification method may be based on a CNN model.

The processor 1400 may sample the preset number of frames from frames constituting the video in which an image of the user’s body is captured and extract feature vectors for the sampled frames. Thereafter, the processor 1400 may fuse the feature vectors extracted from the sampled frames to generate a feature vector for the user’s body motion data. The processor 1400 may predict the occurrence and progression stages of the degenerative neurological diseases by performing a CNN- based classification process of the user’s motion data acquired through capturing into normal or abnormal groups and a detailed classification process of the abnormal group by progression stage.

According to an embodiment, the processor 1400 may further utilize the user’s uttered voice to improve the accuracy of predicting the occurrence and progression stages of the degenerative neurological diseases. For example, for the patients with degenerative neurological diseases caused by vascular disease, the presence or absence of degenerative neurological diseases may be determined solely using uttered voice data due to asymmetry in facial muscle movement.

To this end, the training device 1000 of the present invention may further include a microphone (not illustrated) for receiving the user’s uttered voice. However, the microphone may be provided separately from the training device 1000 and connected to the outside, or may be included in the display device 3000.

The processor 1400 may recognize the user’s uttered voice for specific words or sentences according to the degenerative neurological disease prevention training program through the microphone.

For example, when the degenerative neurological disease prevention training program instructs a user to say specific words or sentences, such as “Rock/Paper/Scissors,” “one/two/three,” or “up/down/left/right,” the processor 1400 may perform control such that the user’s uttered voice is recorded for a certain period of time (e.g., 1.5 seconds).

When it is assumed that the instruction to speak “Scissors,” is performed, the user utterance may be performed in various forms, such as the recorded uttered voice being an incorrect response of “Rock” or “Paper” instead of the instructed “Scissors,” the recorded uttered voice being the correct answer “Scissors” but with slurred pronunciation or a delayed reaction time, or a discrepancy between facial movement and utterance time, and characteristic aspects may commonly occur in the patients with degenerative neurological diseases.

The recorded uttered voice may be converted into WAV files and trained through the CNN. In this case, a preprocessing operation may be performed to extract feature data expressed as a power spectrum of the voice data using the Mel spectrum or the MFCC algorithm.

Based further on the results of comparing the recognized uttered voice data with the voice data of the same words or sentences of the patients with degenerative neurological diseases or the patients suspected of having the degenerative neurological diseases according to the neurological disease prevention training program pre-stored in the external server 2000, the processor 1400 may predict the occurrence and progression stages of the user’s degenerative neurological diseases.

When additional training is performed on data related to the facial muscle movements (such as movements of the mouth or around the mouth) captured through the camera 1200 while the user voice is uttered, the prediction accuracy for the occurrence and progression stages of the degenerative neurological diseases may be further improved.

For example, in the case of the patient with degenerative neurological diseases caused by vascular disease, the occurrence and progression stages of the degenerative neurological diseases may be more accurately predicted by training the asymmetry of the facial muscle motion and the voice data together. Thereafter, the processor 1400 may output the prediction results to the display device 3000 and control transmission to the medical institution device through the training communication unit 1100. The medical institution device may receive the prediction results and call or monitor the patients with the degenerative neurological diseases.

Meanwhile, the processor 1400 may further predict the occurrence of the user’s degenerative neurological diseases, and the types and the progression stages of the occurring degenerative neurological diseases based on the user information (sex, age, family history, pre-existing diseases, medical history, drinking history, etc.) received from the input unit.

For example, the response speed and motion size vary depending on the sex and age, and many elderly patients with dementia also suffer from coexisting diseases such as diabetes, hypertension, and hyperlipidemia. In addition, patients with a history of cerebrovascular disease, head trauma, or alcohol problems are more likely to experience memory impairment. Therefore, by further learning the user information, the occurrence, types, and progression of degenerative neurological diseases may be more accurately predicted.

In addition, the processor 1400 may adjust the type or difficulty of the degenerative neurological disease prevention training program based on the received user information.

Meanwhile, the training input unit 1500 according to another embodiment of the present invention may receive additional behavioral characteristic information further including at least one of the user’s occupation, hobbies, and lifestyle pattern.

Here, the lifestyle pattern may include information such as the user’s wake-up time, meal times, outdoor activity time, type of outdoor activity, and bedtime.

The processor 1400 may determine the type of degenerative neurological disease prevention training program or motion commands to be provided to the user based on the behavioral characteristic information received from the training input unit 1500.

For example, when the user’s occupation is classified as an occupation that primarily involves working on a computer and does not require much physical activity, the processor 1400 may determine to provide unfamiliar motion commands that utilize the unused body parts.

In addition, the processor 1400 may determine to provide unfamiliar or vulnerable motion commands to the user based on the information such as the user’s hobbies or lifestyle pattern, thereby enhancing the effect of the program.

Meanwhile, FIG. 13 is a block diagram schematically illustrating a configuration of the external server according to another embodiment of the present invention.

As illustrated in FIG. 3, the server 2000 includes a program providing unit 2100, a motion data learning unit 2200, a prediction and diagnosis unit 2300, an evaluation score calculating unit 2400, and a reward providing unit 2500.

The program providing unit 2100 is configured to provide the degenerative neurological disease prevention training program to the training device 1000. The training device 1000 may download, store, and execute the degenerative neurological disease prevention training program from the external server 2000. However, the program providing unit 2100 may also provide a service that allows the degenerative neurological disease prevention training program to be executed through the cloud without being downloaded to the training device 1000.

The motion data learning unit 2200 is configured to receive a user’s captured video or motion data extracted from the user and cumulatively train the received motion data through the machine learning. The user’s captured video may undergo image processing using the computer vision technology.

The prediction and diagnosis unit 2300 is configured to compare the motion data extracted from the user with the motion data of the patients with degenerative neurological diseases or the patients suspected of having degenerative neurological diseases that have been cumulatively learned, and to predict the occurrence and progression stages of the user’s degenerative neurological diseases based on the comparison results.

That is, the prediction of the occurrence and progression stages of the user’s degenerative neurological diseases may be performed by the external server 2000.

Meanwhile, the training device 1000 may display, through the display device 3000, a captured video of a user who is provided with the degenerative neurological disease prevention training program and multiple other users connected to the external server, in the synapsology brain activation program in which multiple users participate.

In this case, the evaluation score calculating unit 2400 may calculate and sum the evaluation scores of the motion data extracted according to each action command provided in the degenerative neurological disease prevention training program.

Here, the evaluation score may be calculated by considering the user’s action accuracy, body balance, reaction time, duration, sense of rhythm, and voice accuracy.

The reward providing unit 2500 may provide a reward based on the calculated evaluation score to the user and multiple other users. Here, the rewards may be converted to cash, points that may be used to purchase goods or services, etc.

In particular, the reward providing unit 2500 may compare the user’s previously extracted motion data and prediction information with the currently extracted motion data and prediction information to determine whether the degenerative neurological diseases have improved, and when the degenerative neurological diseases have improved, may provide the user with a reward based on the improvement.

For example, when the progression stage of the degenerative neurological diseases has decreased to a less dangerous level, the reward providing unit 2500 may provide a reward based on the decreased progression stage.

In addition, according to an embodiment, the evaluation score calculating unit 2400 may allow the motion command given in the degenerative neurological disease prevention training program to be shown only to the user and one of multiple other users, and may calculate and sum the evaluation scores of the motion data extracted according to the motion command after the motion command is sequentially transmitted from the user to whom the motion command was shown to the remaining users. The reward providing unit 2500 may provide the reward according to the calculated evaluation scores to the user and multiple other users.

For example, after the motion command is shown to a first user, the first user may transmit the motion command to a second user by performing the motion according to the motion command. In this case, the motion video of the first user is shown only to the second user. The second user may transmit the received motion command to a third user. In this case, the motion video of the second user is shown only to the third user. The third user may transmit the received motion command to a fourth user. In this case, the motion video of the third user is shown only to the fourth user. The fourth user may perform the motion according to the motion command.

The transmission of the operation commands may be repeated in a manner that starts again from the second user and is ultimately transmitted to the first user.

In this way, even without a separate instructor, the evaluation scores for the motion data of the first to fourth users may be calculated, and the rewards may be provided to a user with the highest score, or the rewards according to the obtained evaluation scores may be provided to the user and multiple other users.

Meanwhile, the type or difficulty of the motion commands provided within the degenerative neurological disease prevention training program may vary depending on the user’s performance level.

For example, when the user fails to properly perform the motion according to the given operation command, a motion command of a somewhat easier difficulty level may be provided.

In particular, when the comparison results indicate that the user is suspected of having or is in the process of developing the degenerative neurological diseases, the processor 1400 may verify the prediction results by having the user re-execute the degenerative neurological disease prevention training program or the motion command one difficulty level lower.

FIG. 16 is a flowchart for schematically describing a method of preventing and diagnosing degenerative neurological diseases according to another embodiment of the present invention.

First, the pre-stored degenerative neurological disease prevention training program is output through a display device (S610). In this case, the neurological disease prevention training program may be based on the synapsology brain activation program.

Thereafter, an image of the user is captured and recognized through the camera (S620).

Thereafter, the motion data according to the user’s degenerative neurological disease prevention training program is extracted (S630). Here, the motion data may include at least one of the motion accuracy, body balance, reaction time, duration, rhythm, and voice accuracy according to the motion commands of the degenerative neurological disease prevention training program.

In this case, the motion command of the degenerative neurological disease prevention training program to be provided to the user may be determined based on the behavioral characteristic information, which further includes at least one of the user’s occupation, hobbies, and lifestyle pattern.

In addition, the motion data extraction may also be performed by detecting the preset landmark on the user’s body using the MobileNet model to extract the body coordinates, including the user’s face and palms.

Thereafter, the extracted motion data is compared with the motion data of the patients with degenerative neurological diseases or the patients suspected of having degenerative neurological diseases according to the degenerative neurological disease prevention training program, which is cumulatively learned through the machine learning and stored on the external server (S640).

Thereafter, based on the comparison results, the occurrence and progression stages of the user’s degenerative neurological diseases are predicted (S650).

In this case, the occurrence of the user’s degenerative neurological diseases, and the types and the progression stage of the occurring degenerative neurological diseases may be predicted based further on the user information including the user’s sex, age, family history, pre-existing diseases, medical history, drinking history, etc.

Thereafter, the prediction results are output to the display device and transmitted to the remote medical institution device (S660).

Meanwhile, the external server may compare the user’s previously extracted motion data and prediction information with the currently extracted motion data and prediction information to determine whether the degenerative neurological diseases have improved, and when the degenerative neurological diseases have improved, may provide the user with the rewards based on the improvement.

In addition, according to another embodiment of the present invention, the video captured by the user who is provided with the degenerative neurological disease prevention training program and multiple other users connected to the external server may be displayed on the display device. In this case, the external server may calculate and sum the evaluation scores of the motion data extracted according to each motion command given within the degenerative neurological disease prevention training program, and provide rewards based on the calculated evaluation scores to the user and multiple other users.

In addition, according to an embodiment, the motion command given in the degenerative neurological disease prevention training program may be shown only to the user and one of multiple other users and is sequentially transmitted from the user to whom the motion command is shown to the remaining users, and then the evaluation scores of the motion data extracted according to the motion command are each calculated and summed so that the reward according to the calculated evaluation scores may be provided to the user and multiple other users.

According to various embodiments of the present invention as described above, by predicting the occurrence and progression stages of the degenerative neurological diseases based on the machine learning while allowing the users to perform the training for the prevention of degenerative neurological diseases in a fun and effective manner, it is possible to enable the early diagnosis and prevention of the degenerative neurological diseases such as dementia.

Meanwhile, the methods for preventing and diagnosing degenerative neurological diseases according to the various embodiments described above may be implemented as programs and stored in various recording media. That is, the computer program capable of executing the methods for preventing and diagnosing degenerative neurological diseases described above by being processed by various processors may be stored in a recording medium and used.

For example, there may be provided a non-transitory computer-readable medium in which a program is stored, in which the program performs the operations of: i) outputting a pre-stored degenerative neurological disease prevention training program through a display device, ii) recognizing a user by capturing an image of the user through a camera, iii) extracting motion data according to degenerative neurological diseases of a user prevention training program, iv) comparing the extracted motion data with motion data of a patient with degenerative neurological diseases or a patient suspected of having degenerative neurological diseases according to the degenerative neurological disease prevention training program that is cumulatively learned by machine learning and stored in an external server, v) predicting the occurrence and progression stages of the user’s degenerative neurological diseases based on the comparison results, and vi) outputting the prediction results to the display device and transmitting the prediction results to a remote medical institution device.

The non-transitory computer-readable medium is not a medium in which data is stored for a while, such as a register, a cache, a memory, or the like, but is a medium in which data is semi-permanently stored and that is readable by a device. In detail, the various applications or programs described above may be stored and provided in the non-transitory computer readable media such as a compact disc (CD), a digital versatile disk (DVD), a hard disk, a Blu-ray disc, a Universal Serial Bus (USB), a memory card, and a read only memory (ROM).

According to the system and method for diagnosing and alleviating degenerative neurological diseases according to an embodiment of the present invention, by more thoroughly diagnosing the conditions of the user, i.e., the dementia patient, and providing the customized training program accordingly, it is possible to achieve more effective improvement of dementia.

In addition, according to the system and method for diagnosing and alleviating degenerative neurological diseases according to an embodiment of the present invention, by confirming the user’s image and voice through the login screen provided upon the user login and converting the user’s image and voice into data, it is possible to more thoroughly diagnose the conditions of the dementia patient.

In addition, according to the system and method for diagnosing and alleviating degenerative neurological diseases according to an embodiment of the present invention, by providing a user interface (UI) screen that can be more easily used by the elderly, it is possible to lower the accessibility barrier for the elderly.

Effects of the present invention are not limited to the above-described effects, and effects that are not mentioned will be clearly understood by those skilled in the art to which the present invention pertains from the present specification and the accompanying drawings.

While exemplary embodiments have been shown and described above, it will be apparent to those skilled in the art that modifications and variations can be made without departing from the spirit and scope of the present invention as defined by the appended claims.

What is claimed is:

1. A system for diagnosing and alleviating degenerative neurological diseases through a program provided to a user, the system comprising:
an input unit that receives a user’s personal information and medical history information, and voice information uttered by the user;
a memory unit that stores a first video, which is a video of an expert’s motion and text to be uttered by the user;
a camera unit that acquires a second video, which is a video of the user’s motion;
a display unit that outputs the first video and the second video; and
a control unit that compares accuracy, balance, and reaction speed of the motion output in the second video based on the first video and compares accuracy and pronunciation speed using the voice information to calculate a grade of the user’s degenerative neurological diseases.

2. The system of claim 1, wherein the control unit controls a login screen to be output on the display unit before the first video is output,
the login screen includes an identification screen that identifies the user’s specific body part and an utterance guidance screen that confirms the user’s utterance, and
the control unit acquires login information, which is the user’s image information and the user’s utterance information, through the login screen to calculate the grade of the degenerative neurological diseases.

3. The system of claim 2, wherein the memory unit stores reference information for calculating a numerical value for the login information by comparing the login information, and
the control unit quantifies the login information based on the reference information to calculate the grade of the user’s degenerative neurological diseases.

4. The system of claim 3, further comprising a communication unit that transmits the personal information, the medical history information, the second video, the voice information, and the login information to a central server,
wherein the control unit controls the communication unit to request pre-stored login information from the central server based on the reference information when the numerical value of the login information is greater than or equal to a preset value, and
the control unit calculates the grade of the user’s degenerative neurological diseases based on the pre-stored login information using at least one of a multifactorial variable regression analysis model and an artificial intelligence time series inference model.

5. The system of claim 4, wherein the control unit is configured to:
cause the identification screen to be displayed with priority over the utterance guidance screen when the login screen is output on the display unit, and
output the utterance guidance screen on the display unit based on the reference information when a numerical value of the user’s acquired image information is greater than or equal to the preset value.

6. The system of claim 5, wherein the memory unit pre-stores a guide screen, which is a user interface (UI) screen for providing guidance on membership registration, and
the control unit controls the display unit to output the guide screen when there is no information corresponding to the user’s acquired image information.

7. A method of diagnosing and alleviating degenerative neurological diseases through a program provided to a user, the method comprising:
receiving, by an input unit, a user’s personal information and medical history information;
outputting, by the control unit, a first video, which is a video of an expert’s motion and voice, and a second video, which is a video of the user’s motion acquired from a camera unit, on the display unit;
acquiring, by the input unit, the user’s voice information based on the first video;
calculating, by the control unit, a grade of the user’s degenerative neurological diseases based on the second video and the voice information; and
outputting, by the control unit, a training video corresponding to the calculated grade on the display unit.
